(19) United States
(12) Patent Application Publication
(10) Pub. No.: US 2026/0335686 A1
(43) Pub. Date: Oct. 29, 2026

(54) DEVICES, SYSTEMS, AND METHODS FOR PAIN SUPPRESSION THROUGH TARGETED PERIPHERAL NERVE APPLICATION OF FOCUSED ULTRASOUND

(71) Applicant: THE BOARD OF TRUSTEES OF THE LELAND STANFORD JUNIOR UNIVERSITY, Stanford, CA (US)

(72) Inventor: Thomas Anthony Anderson, Menlo Park, CA (US)

(21) Appl. No.: 19/660,060

(22) Filed: Apr. 27, 2026

Related U.S. Application Data
(60) Provisional application No. 63/795,897, filed on Apr. 28, 2025.

(51) Int. Cl. A61N 7/00

(52) U.S. Cl. CPC A61N 7/00; A61N 2007/0026

(57) ABSTRACT

Devices, systems, and methods are provided for relieving peripheral nerve pain in a subject, e.g., to cause reversible inhibition of nociceptive fibers with or without substantial inhibition of peripheral nerve sensory and motor fibers of the target nerve. In one example, the device includes a surface configured for placement against a subject's skin, an ultrasound imager configured to transmit signals from the surface into the subject's body and receive reflected signals from the body, and an array of transducers configured to deliver focused ultrasound from the surface into the body. A controller is coupled to the imager to process the reflected signals to identify a target nerve within the body and coupled to the array to control delivery of the focused ultrasound to the target nerve to relieve pain.

### RELATED APPLICATION DATA

The present application claims benefit of co-pending U.S. provisional application Ser. No. 63/795,897, filed Apr. 28, 2025, and is related to co-pending U.S. application Ser. No. 18/542,494, filed Dec. 19, 2023, which is a continuation of International Application No. PCT/US2022/033687, filed Jun. 15, 2022, which claims benefit of U.S. provisional applications Serial Nos. 63/210,864, filed Jun. 15, 2021, and 63/314,143, filed Feb. 25, 2022, the entire disclosures of which are expressly incorporated by reference herein.

### STATEMENT REGARDING FEDERALLY SPONSORED RESEARCH AND DEVELOPMENT

This invention was made with Government support under contract EB035005 awarded by the National Institutes of Health. The Government has certain rights in the invention.

### TECHNICAL FIELD

The present application is related to devices, systems, and methods for delivering focused ultrasound into a subject's body and, more particularly, to devices, systems, and methods for relieving acute or chronic pain using focused ultrasound targeting one or more nerves of a subject having pain, e.g., to cause reversible inhibition of nociceptive fibers with or without substantial inhibition of peripheral nerve sensory and motor fibers of the target nerve.

### BACKGROUND

Subjects may experience moderate to severe acute pain after surgery and trauma, which may result in adverse patient outcomes, including opioid dependence and chronic pain. For example, a patient may experience acute pain, e.g., due to surgery or an injury, or the pain may be chronic pain, e.g., due to conditions such as complex regional pain syndrome (“CRPS”) type 1 and 2, phantom limb pain, trigeminal neuralgia, Bell's palsy, intercostal pain, post-herpatic neuralgia, endometriosis, and neuroma.

Current analgesic strategies, include systemic analgesics/opioids and invasive local anesthetic-based peripheral nerve blocks (“PNBs”). Systemic analgesics have dose limiting side effects and opioids additionally have major short-and long-term adverse effects. PNBs have major limitations, including their invasiveness, motor and non-pain sensory blockade, infection risk, and short duration of action relative to the duration of pain after injuries.

Accordingly, devices and methods for improving pain management, e.g., for non-drug-based pain suppression, would be useful.

### SUMMARY

The present application is related to devices, systems, and methods for delivering focused ultrasound into a subject's body. More particularly, devices, systems, and methods are provided for relieving pain using focused ultrasound to target one or more nerves of a subject having pain, e.g., to cause reversible inhibition of nociceptive fibers with or without substantial inhibition of peripheral nerve sensory and motor fibers of the target nerve.

Focused ultrasound (“FUS”) has emerged as a non-invasive, drug-free modality capable of reversibly inhibiting peripheral nerve activity by applying acoustic energy to targeted nerve regions. However, three barriers have limited clinical translation:
1. Lack of defined acoustic parameters that achieve a) reversible inhibition of all peripheral nerve fibers and/or b) selective, reversible inhibition of only nociceptive (Aδ and C) fibers while sparing motor and non-pain sensory functions;
2. Inefficient coupling between the FUS transducer and patient anatomy, leading to variable targeting and energy transmission; and
3. Complex operation, requiring expert users to identify and align nerves and select appropriate parameters.

The systems and methods herein may address one or more of these barriers by defining the FUS biophysical parameter space, developing a height-adjustable coupling interface, and/or integrating an intelligent software system for guided, operator-independent use. In particular, prior efforts did not have specific FUS parameters for noninvasive application to peripheral nerves that resulted in reversible inhibition of all peripheral nerve fibers or only the nociceptive fibers for weeks and did not have evidence that reversible peripheral nerve inhibition prevented or treated acute pain.

As described further elsewhere herein, systems and methods are provided that include validated acoustic parameter sets that achieve reversible blockade lasting weeks (not minutes), selective nociceptive inhibition, with or without motor or non-pain sensory loss. These parameters may provide reproducible, long-duration, reversible blockade, unlike previous systems focused on transient neuromodulation or irreversible neurolysis.

In accordance with one exemplary device, a device is provided with diagnostic and focused ultrasound transducers co-aligned, e.g., with axes that intersect at a desired focal zone or location. For example, based on knowledge of patient anatomy and the peripheral nerve of interest based on the patient's injury or surgery, the diagnostic ultrasound imager is used to locate the peripheral nerve. For this device, once the peripheral nerve is located, the nerve is positioned in a desired focal zone, e.g., the −3 dB focal zone (high energy zone) of the FUS transducer.

The devices, systems, and methods herein may also include a height-adjustable, anatomically adaptive coupling interface that maintains co-registration between diagnostic and therapeutic transducers, compensates for nerve depth variability, and/or minimizes reflection and near-field energy loss. Once the correct parameters and placement are achieved, the system ensures reproducibility via closed-loop control, integrating: real-time imaging of nerve position, visualization of the −3 dB focal zone relative to anatomy, continuous monitoring of delivered acoustic energy and coupling quality, safety interlocks for misalignment or overheating.

In accordance with one example, focused ultrasound may be delivered to relieve pain without damaging the target nerve or other tissues. The pain being treated may be acute pain, with indications including but not limited to acute surgical pain in which the pain is resolved through specific peripheral nerve blockade. The pain may otherwise be chronic pain, with indications including but not limited to complex regional pain syndrome (CRPS) type 1 and 2, phantom limb pain, trigeminal neuralgia, Bell's palsy, intercostal pain, post-herpatic neuralgia, endometriosis, and neuroma.

The use of focused ultrasound for neuromodulation has demonstrated success in a wide range of applications, from the removal of amyloid plaques in Alzheimer's to the disruption of electrographic seizure activity. Most prior work with low-intensity focused ultrasound has been used for transcranial excitation or inhibition of neural structures, such as direct activation of brain regions using transcranial focused ultrasound pulses or induced motor activity in response to transcranial focused ultrasound. High intensity ultrasound has been previously used for ablation of tissues, such as solid tumors. There has been limited prior work using low-intensity ultrasound for peripheral nervous system neuromodulation, such as partial vagus nerve inhibition.

However, there currently exists no device or procedure to use focused ultrasound for peripheral nervous system neuromodulation, including one or both of suppression and excitation of peripheral nerves, for the purposes of acute and chronic pain suppression. The majority of pain management is currently accomplished through narcotics, which have a range of side effects. Focused ultrasound may be used as an alternative, noninvasive and non-drug-based method for pain suppression.

In accordance with one example, a device is provided for relieving pain in a subject that includes a housing including a surface configured for placement against a subject's skin; an ultrasound imager configured to transmit signals from the surface into the subject's body and receive reflected signals from the body; an array of transducers configured to deliver focused ultrasound from the surface into the body; and a controller coupled to the imager to process the reflected signals to identify a target region, e.g., a peripheral nerve, within the body and coupled to the array to control delivery of the focused ultrasound to the target region to relieve pain.

In accordance with another example, a method is provided for relieving pain in a subject that includes placing a contact surface of an ultrasound device against the subject's skin such that an array of ultrasound transducers of the device are acoustically coupled to the skin; activating an ultrasound imager of the device to transmit signals from the surface into the subject's body and receive reflected signals from the body to identify a target region, e.g., a peripheral neve, within the body; and delivering focused ultrasound from the array from the surface into the body to the target region to relieve pain.

In one example, the devices herein may include one or more acoustic transducers coupled to a controller such that the device is configured to transmit low-intensity ultrasound, e.g., with an intensity up to five hundred Watts per square centimeter (i.e., between 0 -500 W/cm²), e.g., at frequencies between about one kilohertz and ten megahertz (1 KHz-10 MHz). In another example, the devices may be configured to transmit high-intensity ultrasound, e.g., with an intensity between about five hundred and two thousand Watts per square centimeter (500-2000 W/cm²), e.g., at frequencies between about one kilohertz and ten megahertz (1 KHz-10 MHz).

In some examples, the ultrasound energy is emitted by the array of ultrasound transducer(s) substantially continuously. In other examples, the ultrasound energy is emitted intermittently. In yet other examples, the ultrasound energy is emitted in pulsatile form.

In various examples, invasive or non-invasive devices may be provided. In one example, a noninvasive ultrasound device may be provided that includes one or more focused ultrasound transducers that may be applied directly onto a subject's epidermis in order to target a specific peripheral nerve. The location on the epidermis for application of the device relative to a target peripheral nerve may be determined through image guidance, such as through ultrasound imaging or magnetic resonance imaging (“MRI”).

In one example, transducer location may be determined prior to or during application of the transducer and, optionally, the focal distance of the ultrasound transducer may be calibrated based on image guidance. For example, the controller may identify different tissue structures between the skin and the target nerve based at least in part on signals from the imaging element, e.g., identifying skin thickness and/or the location and/or thickness of fat layers and muscle layers, and modify the signals to the focused ultrasound (“FUS”) transducer to consider the differing ultrasound attenuation of the tissues along the path to the target nerve to enhance focusing the acoustic energy. Once the correct position and/or focal distance are determined, the transducer may be directly applied onto the epidermis with or without coupling material, such as gel, between the transducer and epidermis. Focused ultrasound energy may then be transmitted to the targeted peripheral nerve through the skin and intervening soft tissue.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 shows an example of a device for delivering focused ultrasound into a subject's body to a target nerve to suppress pain.

FIG. 2 is a schematic showing exemplary components that may be included in the device of FIG. 1.

FIG. 3 shows another example of a device including an adjustable ultrasound imaging element.

FIG. 4 is a cross-sectional view of yet another exemplary device for delivering focused ultrasound into a subject's body.

FIG. 5 shows the device of FIG. 4 being positioned on a subject's body to position a focal zone of the device at a target nerve.

FIGS. 6A and 6B show an exemplary device used to deliver focused ultrasound during rat model studies.

FIG. 6C is an exemplary ultrasound image showing a rat's sciatic nerve overlayed with a focal zone of the device of FIGS. 6A and 6B.

FIGS. 7A-10C are graphs showing behavioral test results from rat model studies.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

The term “substantially” is used throughout this document to indicate variations in the thus qualified terms. These variations are variations that do not materially affect the manner in which the invention works and can be due, for example, to uncertainty in manufacturing processes or to small deviations from a nominal value or ideal shape that do not cause significant changes to the invention. Also, the terminology “proximal” and “distal” refers to a position relative to an operator using the present disclosure on a patient. For example, distal elements are closer to an intervention site, e.g., a patient's skin, and proximal elements are closer to an operator, for example, a doctor or other caregiver using the devices herein.

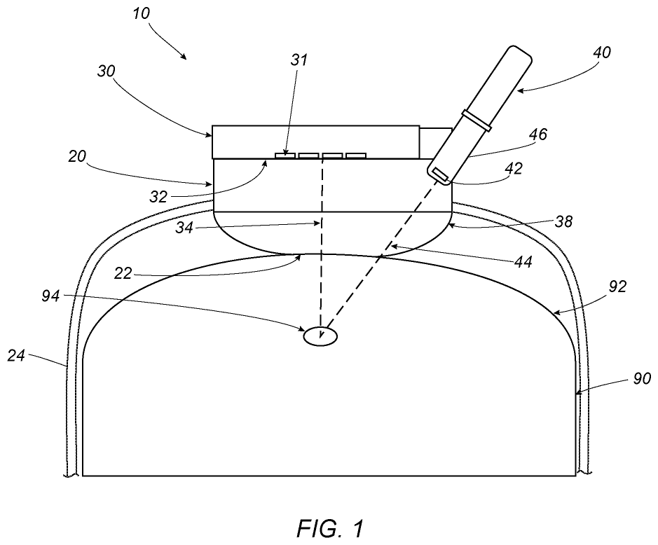

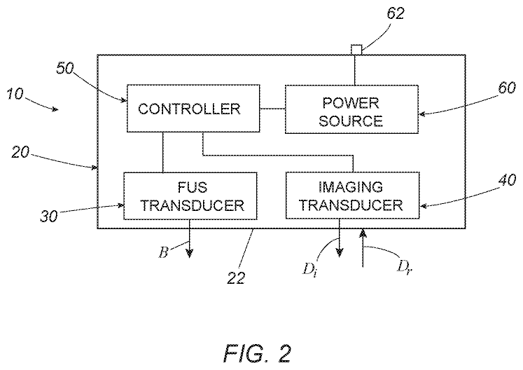

Turning to the drawings, FIG. 1 shows an example of a device 10 for relieving peripheral nerve pain in a subject's body 90 that includes a housing 20 containing one or more components for operating the device 10, e.g., a FUS transducer array 30, an imager or locating element 40, a controller 50, and a power source 60, as shown in FIG. 2. The housing 20 may include a contact surface 22 configured for placement against the subject's skin 92, e.g., to allow focused ultrasound to be delivered from transducer 30 through the surface 22 into the subject's body 90, as described further elsewhere herein.

Optionally, the housing 20 may be shaped to provide a handheld device, e.g., having a shape to facilitate the subject holding the device 10. For example, the housing 20 itself may be shaped to allow a user to hold the device 10, e.g., having an elongated cylindrical or other shape, and/or one or more handles, grips, or other features (not shown) may extend from the housing 20 to facilitate manipulation of the device 10 during use. Alternatively, the housing 20 (or any of the other devices herein) may be mounted on a movable arm (not shown) that includes a first end or base that may be secured to a surgical table, cart, or other structure, and a second end carrying the housing 20, which may be manipulated by an operator, e.g., to place the contact surface 22 against a subject's skin during a treatment, as described further elsewhere herein.

In addition or alternatively, the housing 20 may include one or more features to facilitate securing the device 10 (or any of the other devices herein) relative to the subject's body 90. For example, the contact surface 22 may include adhesive or other tacky material that may secure the surface 22 to the skin 92 during use, but allow the device 10 to be removed without damaging the skin 92.

In addition or alternatively, one or more straps may be provided on the housing 20 that may be wrapped at least partially around the subject's body. For example, as shown in FIG. 1, a pair of straps 24, e.g., extending from opposite sides of the housing 20 having sufficient length to allow the straps 24 to be wrapped around an appendage, e.g., arm or leg, or the torso of the subject's body during use. Optionally, the straps 24 may include ends with cooperating fasteners, e.g., hook and eye fasteners, snaps, clips, buttons, ties, and the like (not shown), for removably securing the ends together to hold the contact surface 22 against the skin 92. Alternatively, an operator, the subject, or another person may simply hold the housing 20 and press the surface 22 against the skin 92 during use. In a further alternative, if the device 10 is carried on an arm, the arm may have sufficient resistance to movement to hold its position, e.g., when the device 10 is positioned against the patient's skin.

With continued reference to FIG. 1, the FUS transducer array 30 may include a plurality of piezoelectric elements 31 providing an array such that the transducer 30 delivers focused ultrasound along a first axis 34, e.g., to generate a beam that focuses the ultrasonic energy at a desired location along the first axis 34, e.g., at a target nerve 94, as described further elsewhere herein. For example, as shown in FIG. 1, the transducer elements 31 may be mounted adjacent the contact surface 22 such that the elements 31 may be acoustically coupled to the skin 92 contacted by the surface 22 and ultrasonic energy generated by the elements 31 is transmitted along the first axis 34 from the surface 22 into the subject's body 90. In the example shown, the transducer elements 31 are mounted to or adjacent a substantially planar surface 32 in a substantially flat array, although it will be appreciated that the elements 31 may be provided in other arrangements, e.g., having a concave, convex or other arrangement centered on the axis 34 (not shown), as desired. In one example, the elements 31 may be positioned adjacent one another on the planar surface 32, e.g., in a circular arrangement on the surface 32 with one or more annular sets of elements 31, each including multiple elements 31 spaced apart radially relative to one another, or may be provided in a linear array, e.g., including one or more rows, each row including multiple square, rectangular, hexagonal, or otherwise shapes piezoelectric elements (not shown).

An acoustic pad or coupling device 38 may be mounted or otherwise coupled to the planar surface 32 that defines the contact surface 22 of the device 10 (or any of the other devices herein), e.g., to acoustically couple the FUS transducer array 30 to the subject's skin 92 and/or to facilitate pressing the device 10 against the skin 92. For example, the pad 38 may be a bag or other flexible membrane containing acoustic gel, water, or other fluid, foam, or other material filling the space between the planar surface 32 and the contact surface 22 that enhances coupling the transducer 30 to the skin 92. The coupling device 38 may have desired dimensions, e.g., height, width, and depth to allow the device 10 to be manipulated relative to the subject while ensuring the FUS and imaging signals pass through the coupling device 38 into the subject's body. In one example, the coupling device 38 may be a three-dimensional body having a maximum cross-section of about fifteen centimeters (15 cm) in each of its height, width, and depth.

Alternatively, the height between the surfaces 44, 2 may be adjusted using other mechanisms to adjust the depth of focus of the FUS energy. For example, in one alternative, a set of acoustic pads (not shown) may be provided having different dimensions, e.g., different heights, and/or different acoustic properties, and the user may select one for attachment to the device 10. In this alternative, the housing 20 may include one or more connectors adjacent the planar surface 32 that allow the selected pad to be mounted to the housing 20 to provide the contact surface 22. The selected pad may be removed and replaced, as desired, e.g., if multiple locations are to be treated or if a single device is used to treat multiple subjects. This would allow a single device, i.e., with FUS transducer array 30 and imager 40 to be customized by the user during or between uses. The pads may be sealed such that they may be sterilized or otherwise cleaned between uses or as desired.

Optionally, the pad 38 may be adjustable, e.g., along the first axis 34 to allow the focal zone of the ultrasonic energy to be manually adjusted relative to the subject's body 90 when pressed against the skin 92. For example, the membrane may be sufficiently flexible such that the distance between the surfaces 32 and 22 may be adjusted by displacing the gel, fluid, or other pad material outwardly within the membrane, which may expand outwardly to allow displacement without rupturing.

In one example, the coupling device 38 may be a fluid-filled flexible enclosure, e.g., a flexible bag formed from biocompatible material, such as polyurethane and silicone. The fluid may be an incompressible liquid, such as degassed water, gel, and the like. The fluid and enclosure material allow ultrasound signals to pass through the coupling device 38 with minimal attenuation. Alternatively, the coupling device may be a solid pad, e.g., formed from foam, silicone, hydrogel, and the like. Optionally a plurality of pads (not shown) having different thicknesses or heights such that a desired pad may be mounted to the housing 20 to adjust a distance between the imager 40 and the array 30 and the contact surface 22 to adjust a location of the focal zone within the subject's body.

With continued reference to FIG. 1, as shown, the imager 40 includes one or more imaging or diagnostic ultrasound transducer elements 42 configured to transmit incident ultrasound signals Dᵢ, e.g., from the contact surface 22 into the subject's body 90, e.g., with a field of view aligned along a second axis 44, and receive reflected signals Dᵣ from the body 90. In the example shown, the imager 40 may be mounted to the housing 20 such that the field of view of the imaging transducer(s) 42 is offset from the FUS transducer array 30, e.g., laterally relative to the planar surface 32, and angled such that the second axis 44 intersects the first axis 34. For example, the field of view may be aligned to ensure that the focal zone (the high energy zone of the focused ultrasound transmitted by the FUS transducer array 30) is in the middle of the field of view, which may facilitate marking the focal zone on images acquired by the imager 42. The imager 42 may include one or more piezoelectric elements (one shown for simplicity), e.g., provided at a distal end of an imaging housing 46 mounted adjacent the FUS transducer array 30 such that the imaging signals Dᵢ, Dᵣ also pass through the pad 38. Alternatively, the imager 42 may be mounted on the device such that the field of view is aligned along the first axis 34, such that the imaging and focused ultrasound transducers are co-aligned on a common axis. In a further alternative, other imaging devices may be provided capable of generating signals that may be used to identify the target nerve 92 or other tissue of interest.

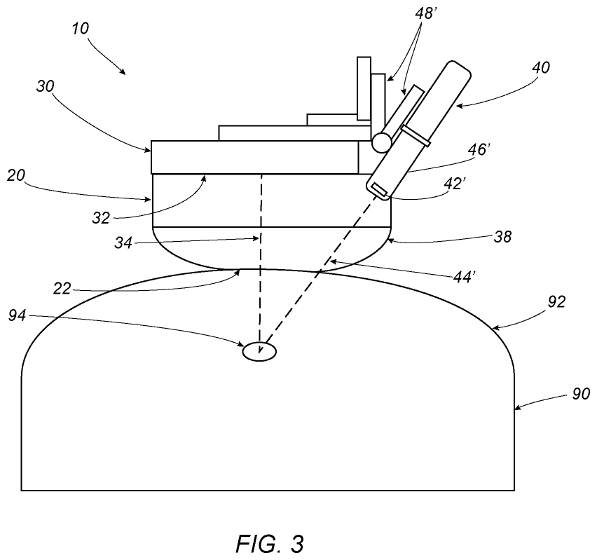

The imager 40 may be fixed relative to the housing 20, e.g., as shown in FIG. 1, such that the intersection angle of the axes 34, 44 is fixed (even if the angles may be otherwise modified electronically as described elsewhere herein). Alternatively, the imager 40 may be movable relative to the housing 20 to change the direction of the second axis 44. For example, as shown in FIG. 3, an imager 40′ may be provided that is coupled to the housing 20 by a mechanism that provides one or more degrees of freedom of movement of the imaging element 40′ relative to the housing 20. In the example shown, the imager 40′ may be mounted to the housing 20 using a hinge 48′ that allows the imager 40′ to pivot relative to the housing 20 about a single axis, e.g., such that the imaging transducer(s) 42 can be pivoted such that the second axis 44 may be substantially parallel to the first axis 34 and may intersect the first axis 34 at an increasing acute angle as the imager 40′ is pivoted, e.g., between about zero and ninety degrees (0-90°). In addition or alternatively, the entire imager 40 may be movable spatially relative to the housing 20, e.g., in one or more directions within a plane parallel to the planar surface 32.

With additional reference to FIG. 2, a controller 50 (including one or more processors, memory, and/or other electronic components, not shown) is coupled to the imager 40 to control delivery of the incident signals Dᵢ and to process the reflected signals Dᵣ, e.g., to generate images and/or identify one or more tissue structures within the body 90. For example, the controller 50 may analyze the reflected signals to identify a target nerve 94 (or other tissue of interest) and determine the depth and/or other spatial information of the nerve 94 relative to the FUS transducer 30.

The controller 50 may also be coupled to the FUS transducer array 30, e.g., to deliver energy to the transducer elements 31 to control delivery of the focused ultrasound to the target nerve 94 to relieve pain. The controller 50 may be coupled to the power source 60, e.g., including one or more batteries, transformers, and/or other components necessary to deliver signals to the transducer elements 31 to cause the elements 31 to generate acoustic energy directed along the first axis 34. As shown in FIG. 2, the controller 50 and power source 60 may be provided within the housing 20. Alternatively, one or both of the controller 50 and the power source 60 may be provided separately from the housing 20, e.g., within a console (not shown) that may be connected to the housing 20 by one or more cables. In this alternative, the console may include a display and/or user interface, if desired, e.g., to activate the device and/or control one or more parameters of operation of the device. For example, a console may be provided on a cart or other structure, e.g., to which an arm carrying the housing 20 is also mounted.

In addition or alternatively, the device 10 may include a connector 62, e.g., to allow the device 10 to be connected to an external power source, e.g., simply plugged into an electrical outlet or an external power source capable of generating the power and/or signals necessary to operate the transducer 30 and/or imaging element 40. When activated, the device 10 may deliver the FUS energy substantially continuously, intermittently, and/or using a desired pulsative form, as desired. Optionally, the device 10 may include a user interface, e.g., touchscreen, set of buttons, and the like (not shown), allowing a user to select desired parameters of the energy delivered, e.g., to modify one or more of intensity, duration, waveform, and the like, if desired.

Optionally, the controller 50 may control one or more of phase, intensity, pulse duration, and frequency of the signals to the piezoelectric elements 31 to generate an ultrasound beam focused at a focal zone that may be directed on the target nerve 94. For example, based at least in part on the location of the target nerve 94 identified by the imaging element 40, the controller 50 may modify the signals to move the depth of the focal zone of the ultrasound beam B and/or to move the focal zone laterally relative to the first axis 34 to focus the acoustic energy on the target nerve 94. In addition, the controller 50 may identify different tissue structures between the skin 92 and the target nerve 94 based at least in part on signals from the imager 40, e.g., identifying skin thickness and/or the location and/or thickness of fat layers and muscle layers. The controller 50 may modify the signals to the elements 31 of the FUS transducer array 30 to consider the differing ultrasound attenuation of the tissues along the path to the target nerve 92 to enhance focusing the acoustic energy. In addition or alternatively, if the imager 40 is movable, the orientation of the imager 40 may be manually adjusted and the controller 50 may modify the signals to adjust the focal zone based at least in part on the location of the target nerve.

In addition or alternatively, the location of the focal zone may be manually adjusted, e.g., using the acoustic pad 38. For example, the distance from the planar surface 32 (and, consequently, the transducer elements 31) to the contact surface 22 may be adjusted by adjusting pressure applied to the pad 38, e.g., to displace gel, fluid, or other material within the pad 38, to provide a desired distance to position the tissue of interest within the focal zone before delivering the FUS energy.

Alternatively, the FUS transducer array 30 may be mounted within the housing 20 such that the FUS transducer array 30 may be moved during use, e.g., using one or more servomotors or other actuators. For example, the housing 20 may be secured to the subject's body 90, e.g., using one or more straps or other features (not shown) and the controller 50 may process signals from the imager 40 to identify one or more regions within the body 90 for treatment.

The transducer array 30 may be activated continuously or intermittently and then moved within the housing 20 and, consequently, relative to the body 90 to move the focal zone. In one example, the transducer array 30 may be movable within a plane parallel to the planar surface 32, thereby allowing the focal zone to be moved laterally relative to tissues within the body 90. For example, if the target treatment region is larger than the focal zone, the transducer array 30 may be moved during a treatment to deliver acoustic energy to the entire region. Similarly, if the target region includes a long nerve, the transducer array 30 may be moved to deliver FUS along a desired length of the nerve. Alternatively, the transducer array 30 may be moved to deliver FUS to multiple regions with acoustic energy delivered to each region for a preset or customized duration.

Optionally, the device 10 may include an output device (not shown) that may be coupled to the controller 50 to assist a user in positioning the focal zone relative to the target nerve. For example, one or more light indicators, a speaker, and the like (not shown) may be provided on the housing 20 that may provide an output when the target nerve is within the focal zone. In addition or alternatively, a display (also not shown) may be provided on the housing 20 (or on a separate console) that may provide images based at least in part on the signals from the imager 40, e.g., identifying the location of the target nerve 94 (and/or other tissue structures) and superimposing a representation of the focal zone to allow the user to manually place the target nerve 94 within the focal zone. For example, the controller 50 may process signals from the imager 40 and present images on the display showing the location of the target nerve 94 relative to the focal zone. As the user moves the device 10, e.g., by pressing the pad 38 and/or moving the contact surface 22 along the skin 92, the location of the target nerve 94 may be monitored as it moves relative to the device 10 until the focal zone overlaps the target nerve 94 in the images, whereupon the user may activate the FUS transducer array 30 to deliver the FUS energy, to relieve the subject's pain.

Thus, the device 10 may be used to temporarily relieve pain by applying FUS energy without damaging the target nerve or, alternatively, may be used to relieve pain indefinitely by causing neuromodulation, ablation, or other at least partial destruction of the nerve.

With additional reference to FIG. 1, during use, the device 10 may be used by a subject or a caregiver for relieving peripheral nerve pain in the subject. Generally, the contact surface 22 may be placed against the subject's skin 92, thereby acoustically coupling the FUS transducer 30 to the skin 92. Optionally, acoustic gel or other material may be applied to the skin 92 and/or to the contact surface 22 before placement to enhance acoustic coupling, if desired.

The imager 40 may be activated, whereupon the controller 50 may cause the imaging transducer(s) 42 to transmit signals Dᵢ from the surface 22 into the subject's body 90 and receive reflected signals Dᵣ from the body 20 to identify a target nerve or other tissue of interest 94 within the body 90. Optionally, the device 10 may be moved along the skin 92 and/or the pad 38 may be pressed or released, as desired, e.g., under guidance from the imager 40. In addition or alternatively, if the imager 40 is movable, the device 10 may be applied to the skin 92 generally over the target nerve 94, and the imager 40 moved to identify the location relative to the FUS transducer 30, and the controller 50 may calibrate the signals necessary to direct the focal zone at the target nerve 94.

Once the target nerve 94 is within a focal zone of the FUS transducer array 30, the FUS transducer array 30 may be activated to deliver focused ultrasound to the target nerve 94, e.g., to relieve pain. Optionally, the FUS transducer array 30 may be manually activated by the user, e.g., after confirming the location of the target nerve 94 (e.g., using an output device on the housing 20 to confirm). Alternatively, the controller 50 may automatically activate the FUS transducer array 30 once the controller 50 confirms that the target nerve 94 is within the focal zone. The FUS energy may be delivered for a preset time or the user may activate the FUS transducer array 30 for a desired amount of time.

In one example, the FUS transducer array 30 may be configured to transmit the focused ultrasound at a frequency of about 1.5 Megahertz. The controller 50 may be coupled to the transducer array 30 to deliver electrical energy from the power source 60 to activate the transducer element(s) 31 to deliver the focused ultrasound during a treatment. In one exemplary configuration, the controller 50 delivers between about 150-300 millivolts to the transducer array 30, thereby generating an average intensity between about 500-2100 Watts per square centimeter (W/cm 2). In another example, the controller 50 delivers between about 200-250 millivolts to the transducer 30, thereby generating an average intensity between about 900-1500 Watts per square centimeter (W/cm2).

The controller 50 may be configured to automatically complete a treatment having a predetermined treatment duration, e.g., between about thirty and ninety seconds (30-90 s), e.g., about sixty seconds (60 s), and/or at a predetermined duty cycle, e.g., between about fifty and one hundred percent (50-100%), or at a one hundred percent (100%) duty cycle. The controller 50 may activate the FUS transducer array 30 continuously during the treatment duration or may activate the FUS transducer array 30 in pulses, e.g., with each pulse having a burst or pulse duration between about twenty five and one hundred milliseconds (25-100 ms), or about fifty milliseconds (50 ms). These parameters may generate a peak-to-peak pressure/intensity at the target nerve between about four and ten Megapascals (4.0-10 MPa) or, more narrowly, between about five and seven Megapascals (5.0-7.0 MPa).

In one specific example, the controller 50 is configured to deliver a voltage to the one or more transducer elements 31 between about 150-300 mV at a duty cycle between about fifty and one hundred percent (50-100%) to generate the focused ultrasound having an average peak-to-peak pressure/intensity between about three to nine MegaPascals (3.0-9.0 MPa) for a treatment duration between about thirty and ninety seconds (30-90 s). Optionally, the controller may deliver the focused ultrasound in a plurality of bursts or pulses, e.g., with each pulse having a pulse duration between about twenty and one hundred milliseconds (25-100 ms) during the treatment duration with the duty cycle (the time the transducer is on during each pulse) being between about fifty and one hundred percent (50-100%) in each pulse.

In another example, the controller 50 is configured to deliver a voltage to the one or more transducer elements between about 200-250 mV at a one hundred percent duty cycle to generate the focused ultrasound having an average peak-to-peak pressure/intensity between about five and seven MegaPascals (5.0-7.0 MPa) for a treatment duration between about sixty seconds (60 s). In this example, the controller 50 may deliver the focused ultrasound in bursts or pulses, e.g., with each pulse having a pulse duration of about fifty milliseconds (50 ms) during the treatment duration with the duty cycle (the time the transducer is on during each pulse) being one hundred percent (100%) in each pulse.

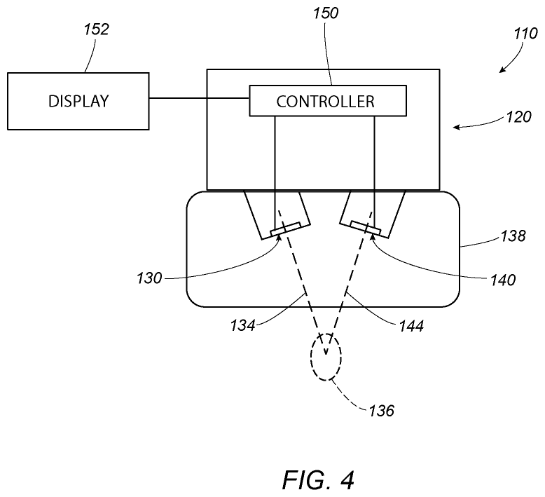

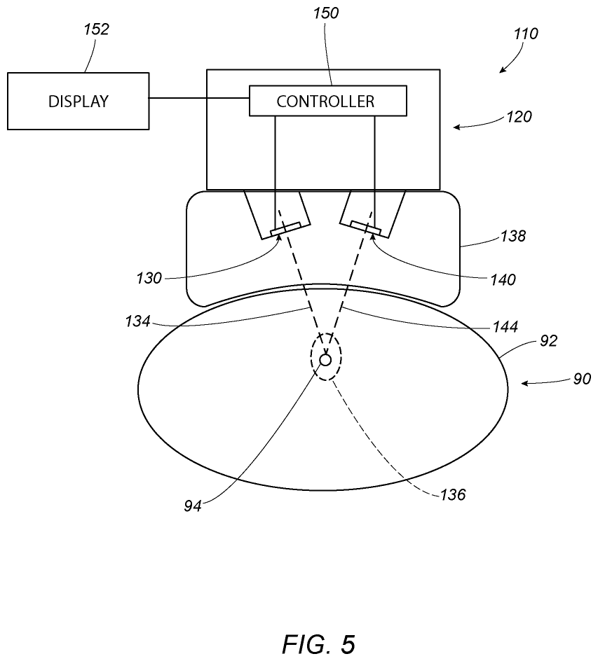

Turning to FIGS. 4 and 5, another example of a device 110 is shown for relieving nerve pain in a subject's body 90 that includes a housing 120 containing one or more components for operating the device 110, e.g., a FUS transducer array 130, an imager 140, and a controller 150, generally configured similar to the other devices herein. Optionally, the housing 120 may also include a power source and/or one or more output devices (not shown) within the housing 120, similar to the other devices herein. Alternatively, the device 110 may be connected to an external power source, e.g., within a console, which may include a display 152 and/or other output devices, a user interface, and the like (not shown). Optionally, in this alternative, the device 110 may be mounted on a movable arm, e.g., mounted to a cart or other structure including the console, to facilitate manipulation and/or positioning the device 110 during use.

In addition, the device 110 includes a coupling device 138 permanently or removably mounted to the housing 120 to provide a contact surface 122 configured for placement against the subject's skin 92, e.g., to allow focused ultrasound to be delivered from the FUS transducer array 130 and imager 140 through the surface 22 into the subject's body 90, also similar to the other devices herein.

The FUS transducer array 130 may include a plurality of transducer elements configured to deliver focused ultrasound (FUS) along a first axis 134 from the housing 120, e.g., into a subject's body 90. The imager 140 may include one or more transducer elements configured to transmit signals along a second axis 144 from the housing 120, e.g., also into the subject's body 90, and receive reflected signals from the body 90. In the example shown in FIG. 4, the FUS transducer array 130 and imager 140 are aligned such that the first and second axes 134, 144 intersect at a desired focal zone 136, e.g., corresponding to a high energy (e.g., −3 dB) zone for the array 130. For example, the FUS transducer array 130 and imager 140 may be fixed relative to the housing 120 and extend from a lower surface of the housing 120 to co-align the axes 134, 144. Alternatively, the array 130 and/or imager 140 may be recessed within and/or flush with a lower surface of the housing 120 if desired, e.g., to facilitate mounting the coupling device 138 to the housing 120.

Similar to the other devices herein, the device 110 may be used to relieve peripheral nerve pain in a subject, e.g., to cause reversible inhibition of nociceptive fibers only without substantial inhibition of peripheral nerve sensory and motor fibers of the target nerve, or to cause reversible inhibition of the nociceptive fibers as well as peripheral nerve sensory and motor fibers of the target nerve. Generally, during use, as shown in FIG. 5, the contact surface 122 may be placed against the subject's skin 92, thereby acoustically coupling the FUS transducer 130 to the skin 92. Optionally, acoustic gel or other material may be applied to the skin 92 and/or to the contact surface 122 before placement to enhance acoustic coupling, if desired.

The device 110 may be positioned until a target nerve 94 is positioned within the focal zone 136, and then the FUS transducer array 130 may be activated to deliver a desired treatment. In one example, images from the imager 140 may be presented on the display 152. The controller 150 may overlay on the images a graphic, e.g., an oval or other shaped outline, crosshair, and the like (not shown) at a location on the images that represents the focal zone 136 of the FUS transducer array 130. For example, given the fixed alignment of the array 130 and imager 140, the controller 150 may process the image signals from the imager 140 to show tissue structures within the images relative to the focal zone 136. The operator may position the device 110 until a target nerve 94 visible in the images is positioned within the graphic, e.g., to center the verve 94 within the graphic and, consequently within the focal zone 136. This positioning may involve moving the device 110 along the subject's skin 92 and/or pressing the device 110, e.g., to compress and/or otherwise displace the coupling device 136 until the nerve 94 is centered in the focal zone 136. The operator may then activate the FUS transducer array 130 to deliver the focused ultrasound to the nerve 94.

For example, once activated, the controller 150 may activate the array 130 using a predetermined set of parameters, i.e., automatically activating the array 130 to deliver a predetermined Total Energy, e.g., based on the following formula:

Total Energy=ISPTA×Duration

where ISPTA is the Spatial-Peal Temporal Average Intensity of the focused ultrasound and Duration is the time the FUS transducers are activated to deliver the focused ultrasound.

In one mode of operation, the device 110 may be activated to cause reversible inhibition of nociceptive fibers without substantial inhibition of peripheral nerve sensory and motor fibers of the target nerve, which may be achieved with a Total Energy between about 30,000 to 40,000, or between about 34,000 and 35,000. In one particular example, the controller 150 may activate the array 130 for a Duration is about sixty seconds (60 s) with the ISPTA between about 500-670 Watts per square centimeter (W/cm2). In another example, the Duration is about sixty seconds (60 s) and the ISPTA is between about 550-600 Watts per square centimeter (W/cm2).

In one example, the array 130 may be configured to deliver the focused ultrasound at a frequency of about 1.5 Megahertz. In this example, the controller may be configured to activate the array 130 at a peak-to-peak pressure of about 4.2 MegaPascals (MPa) at a one hundred percent (100%) duty cycle, with the ISPTA being about 573W/cm2 .

In another example, the device 110 may be activated to cause reversible inhibition of nociceptive fibers, as well as peripheral nerve sensory and motor fibers of the target nerve. To achieve this, the controller may be configured to activate the array to deliver Total Energy of about 85,000 to 87,000, e.g., with the ISPTA between about 1436 Watts per square centimeter (W/cm2) and the Duration at about sixty seconds (60 s).

The devices and methods herein may provide enhanced nerve blockade non-invasively, e.g., unlike conventional needle-based peripheral nerve blocks (PNBs). For such conventional needle-and local anesthetic-based peripheral nerve blocks (PNBs), ultrasound imaging (dUS) is used to identify the nerve and guide a needle to the nerve to deliver one or more drugs, such as opioids and/or anesthetics. The procedure typically involves:
a. Preoperatively, a patient arrives to the preoperative area for an updated history and physical, any final consents, peripheral intravenous catheter placement, and to obtain vital signs;
b. An anesthesiologist sees the patient and gives or has another healthcare provider give sedation prior to the PNB;
c. Using dUS, the peripheral nerve of interest is identified;
d. A needle, connected to a syringe with local anesthetic is inserted and guided so that its tip is near the peripheral nerve; and
e. Local anesthetic is injected, the needle is removed, and patients are monitored for adverse reactions.
In contrast, exemplary procedures involving the devices and methods herein may involve:
a. Preoperatively, a patient arrives to the preoperative area for an updated history and physical, any final consents, peripheral intravenous catheter placement, and to obtain vital signs;
b. An anesthesiologist sees the patient and gives or has another healthcare provider give sedation prior to the PNB;
c. Using dUS, the peripheral nerve of interest is identified;
d. The dUS-FUS system, with a coupling device between the transducers and the patient, will be strapped to the patient's limb to prevent movement of the FZ relative to the PN with any patient movement;
e. Using dUS, the peripheral nerve will be guided into the focal zone on the dUS screen;
f. A validated FUS parameter set will be selected; and
g. FUS will be delivered to the peripheral nerve in a single application

Exemplary configurations for operating the device 100 will now be described. In one example, parameters have been identified that cause reversible suppression of A-delta and C fiber-mediated pain behaviors lasting about three to four (3-4) weeks and motor and non-pain sensory fiber inhibition lasting about four to six (4-6) weeks. The following set of FUS parameters were derived from iterative in vivo studies in rodents and subsequent scaling, which result in reversible inhibition of sensory (both pain and non-pain) and motor fibers:
Frequency: 1.5 MHz
Peak-to-peak pressure: 6.65 MPa
Duty cycle: 100%
Application duration: 60 sec
Spatial peak temporal average intensity (“ISPTA”): ~1436 W/cm²

Based on the studies, all behavioral changes were reversible with full recovery by the subject.

In another example, a reproducible set of FUS parameters has been derived from iterative in vivo studies in rodents and subsequent scaling, which result in reversible inhibition of nociceptive (pain) fibers only without inhibition of peripheral nerve sensory and motor fibers:
Frequency: 1.5 MHz
Peak-to-peak pressure: 4.2 MPa
Duty cycle: 100%
Application duration: 60 sec
Spatial peak temporal average intensity (ISPTA): ~573 W/cm²

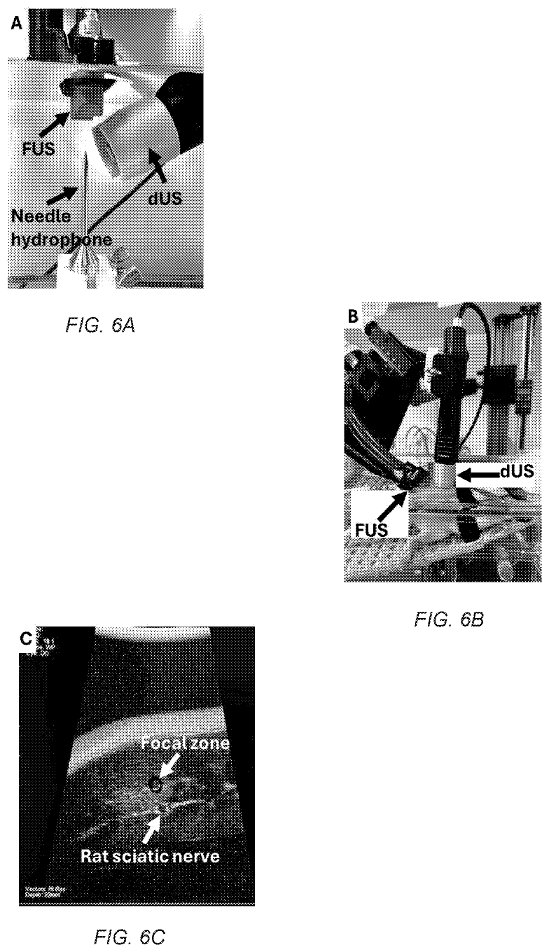

For example, turning to FIGS. 6A and 6B, a rat model series of studies were undertaken that involved acute incisional pain (hind paw, HP, incision) of the subjects. As best seen in FIG. 6A, the device included dUS (imaging) and FUS transducers co-aligned to focus at a common focal zone, which was used to noninvasively deliver energy to the sciatic nerve of rats in a degassed water bath for ease of coupling, e.g., as shown in FIG. 6B. FUS parameters (megapascals, duty cycle, time, burst period) were iteratively adjusted until a parameter set (PS) was determined, resulting in reversible inhibition of PN sensory and motor fibers, as measured with behavioral tests.

Additional tests during the studies were conducted to identify a unique PS (PS1, a single, 1-minute FUS application) that produced selective, reversible inhibition of nociceptive peripheral nerve fibers only. Additionally, in this pain model, control animals with the injury only (HP incision only) sustained thermal hyperalgesia, a surrogate for chronic post-surgical pain; yet, FUS application using PS1, completely prevents the development of this CP behavior. These findings further support the potential of dUS-guided FUS-induced PNB to provide sustained analgesia following injury or surgery while preserving motor and non-pain sensory functions, preventing both acute and chronic pain.

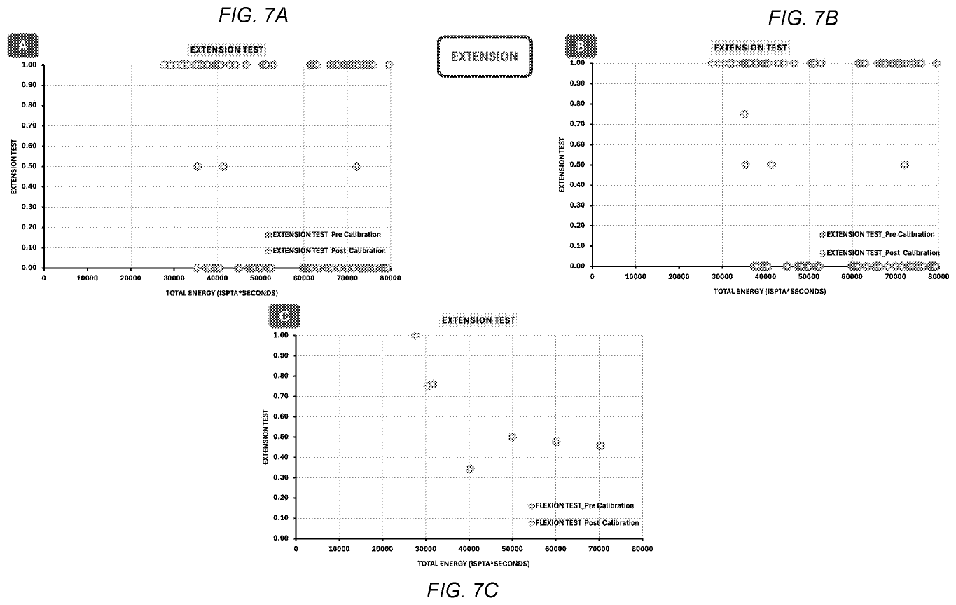

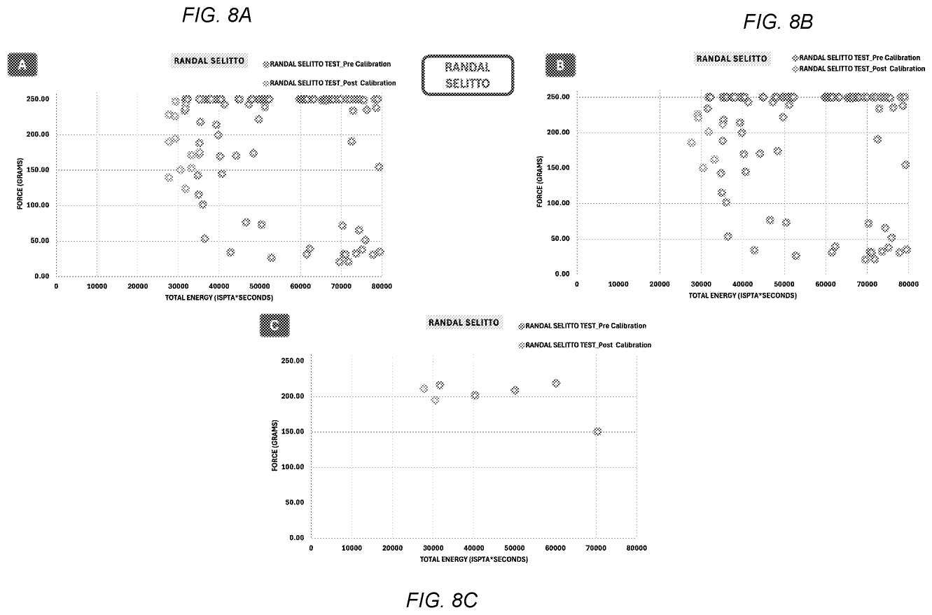

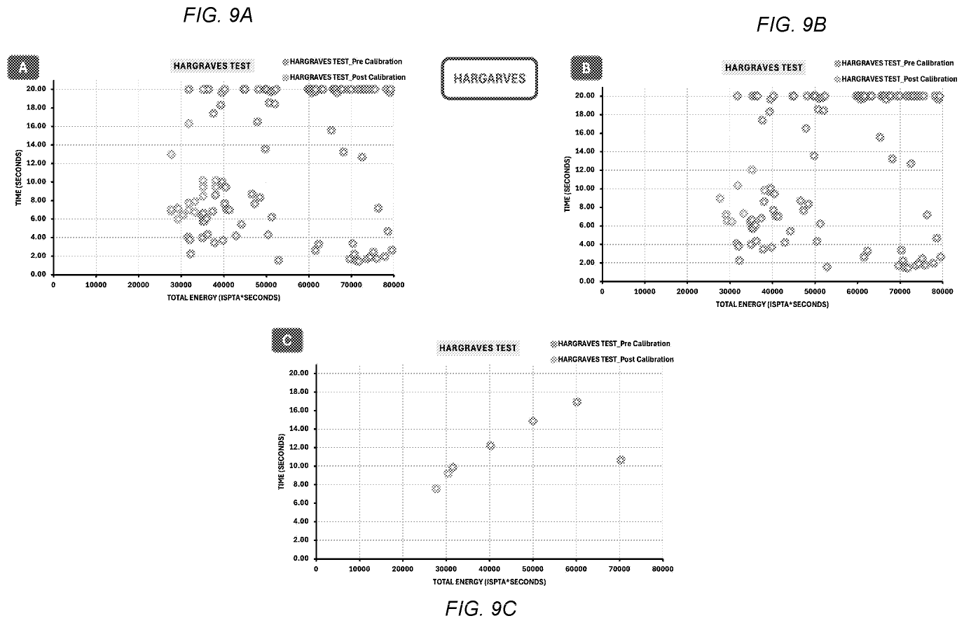

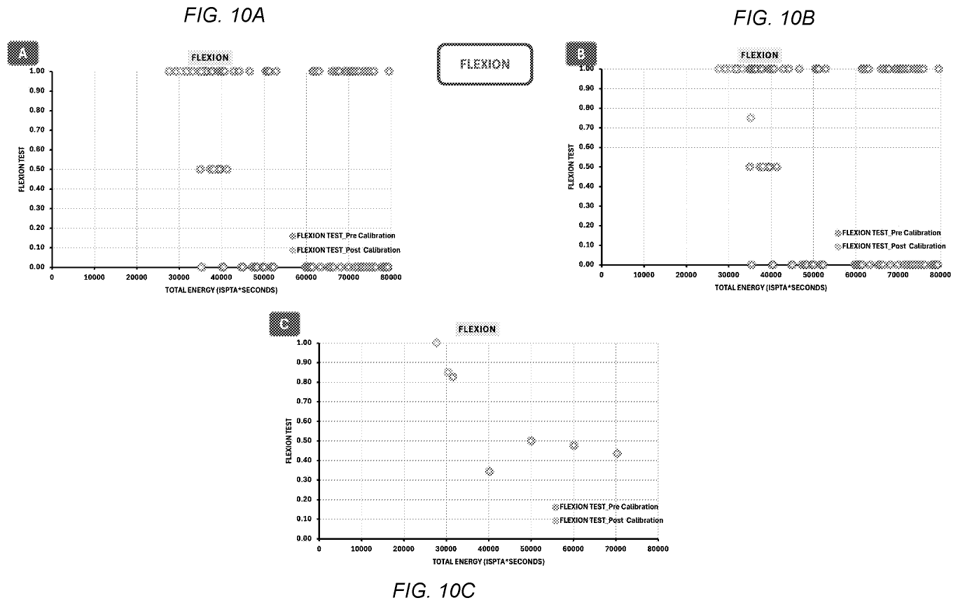

FIGS. 7A-10C show exemplary test results from the studies of the FUS parameter space, i.e., to determine a FUS parameter set that resulted in high values for pain fiber behavioral tests of the subjects. The goal was to confirm that the pain fibers of the subjects were inhibited immediately after delivery of the focused ultrasound to the peripheral nerves but with minimal or no inhibition of motor and non-pain sensory fibers. Motor and non-nociceptive sensory behaviors were minimally/non-statistically significantly affected assessed. For example, HP extension tests (FIGS. 7A-7C) of the hind paws of the subjects tested the subjects'motor function after FUS, and HP flexion tests (FIGS. 10A-10C) tested their motor and non-pain sensory fiber functions. Randall-Selitto tests (FIGS. 8A-8C) and Hargreaves tests (FIGS. 9A-9C) demonstrated immediate inhibition of pain fibers. Mechanical and thermal hypersensitivity were significantly attenuated for four weeks following FUS application and hind paw incision.

Peak negative pressure and ISPTA are related but not fully independent variables. Pressure determines instantaneous mechanical effects, while ISPTA reflects cumulative energy delivery over time. Selectivity depends on their combined effect, along with duty cycle and exposure duration. Correct stimulation of the correct spot is achieved by co-registered diagnostic imaging, focal zone geometry matched to nerve diameter, coupling optimization to prevent beam distortion.

These in vivo rat studies demonstrated that the specified FUS parameters produce reversible inhibition lasting up to three weeks with complete recovery. The prototype systems showed accurate focal targeting and stable coupling to the target nerve was achieved.

The devices, systems, and methods herein may be used to deliver focused ultrasound to a variety of peripheral verves to reduce acute and/or chronic pain, such as the lumbar plexus, femoral, saphenous, obturator, lateral femoral cutaneous, sciatic, posterior tibial, sural, common peroneal, deep peroneal, superficial peroneal, brachial plexus, intercostal brachial, musculocutaneous, median, radial, ulnar, ilioinguinal, iliohypogastric, intercostal, superficial cervical plexus, auriculotemporal, mental, buccal, infraorbital, supraorbital, supratrochlear, greater occipital, great auricular, and lesser occipital nerves. The devices and methods herein may be particularly useful for delivering focused ultrasound to cause reversible inhibition of nociceptive fibers with or without substantial inhibition of peripheral nerve sensory and motor fibers of the sciatic nerve, femoral nerve, and the brachial plexus. Potential conditions that may be treated include phantom limb pain, CRPS, type I & II, acute surgical pain: specific peripheral nerve blockade, neuroma, headaches, trigeminal neuralgia, Bell's palsy, glossopharyngeal nerve, intercostal pain, trigeminal neuralgia, neurolysis (e.g., celiac plexus, superior hypogastric plexus, etc.), endometriosis pain, shingles (e.g., post-herpatic neuralgia), rhizotomy, back pain, rheumatoid arthritis pain, cancer-related pain, and the like.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for relieving peripheral nerve pain in a subject, comprising:
a housing including a surface configured for placement against a subject's skin;
an ultrasound imager on the housing configured to transmit signals from the surface into the subject's body and receive reflected signals from the body;
an array of transducers configured to deliver focused ultrasound from the surface into the body at a frequency of about 1.5 Megahertz; and
a controller coupled to the imager to process the reflected signals to identify a target nerve within the body and coupled to the array to control delivery of the focused ultrasound to the target nerve to relieve pain,
wherein the controller is configured to deliver a voltage to the array between about 150-300 mV at a duty cycle between about fifty and one hundred percent (50-100%) to generate the focused ultrasound.

2. The device of claim 1, wherein the imager and the array are aligned on a common axis.

3. The device of claim 2, wherein the imager has a field of view aligned along an imaging axis and wherein the array is configured to deliver the focused ultrasound along the imaging axis.

4. The device of claim 1, wherein the controller is configured to activate the array for a treatment duration between about thirty and ninety seconds.

5. The device of claim 4, wherein the controller is configured to activate the array in pulses having pulse durations between about twenty five and one hundred milliseconds (25-100 ms) during the treatment duration.

6. The device of claim 1, wherein the array comprises an array of piezoelectric elements mounted adjacent the surface such that the array may be acoustically coupled to the skin.

7. The device of claim 6, wherein the surface comprises a pad configured to couple the array to the skin.

8. The device of claim 7, wherein the pad has an adjustable height along an axis along which the focused ultrasound is delivered.

9. The device of claim 6, wherein the controller is coupled to the array to control one or more of phase, intensity, pulse duration, and frequency of signals to the piezoelectric elements to generate an ultrasound beam focused on the target nerve.

10. The device of claim 1, further comprising a power source coupled to the array and the controller to deliver electrical energy to the array to deliver the focused ultrasound.

11. The device of claim 1, wherein the array is configured to deliver the focused ultrasound along a first axis, and wherein the imager is offset from the one array such that the imaging signals are delivered along a second axis that intersects the first axis at a desired focal zone.

12. The device of claim 11, wherein the imager is movable relative to the housing to adjust an angle of the second axis relative to the first axis.

13. The device of claim 12, further comprising a handle mounted to the imager for adjusting the angle of the second axis.

14. The device of claim 11, wherein the imager and the array are fixed relative to the housing.

15. The device of claim 1, wherein the imager comprises an ultrasound imaging element configured to transmit ultrasound signals into the body and receive reflected ultrasound signals from tissue structures within the body.

16. The device of claim 15, wherein the ultrasound imaging element comprises an array of piezoelectric transducers.

17. The device of claim 1, wherein the controller is configured to calibrate delivery of the focused ultrasound based at least in part on a distance from the array to the target nerve.

18. A device for relieving peripheral nerve pain in a subject, comprising:
a housing including a surface configured for placement against a subject's skin;
an ultrasound imager on the housing configured to transmit signals from the surface into the subject's body and receive reflected signals from the body;
an array of transducers configured to deliver focused ultrasound from the surface into the body at a frequency of about 1.5 Megahertz; and
a controller coupled to the imager to process the reflected signals to identify a target nerve within the body and coupled to the array to control delivery of the focused ultrasound to the target nerve to relieve pain,
wherein the controller is configured to deliver a voltage to the array between about 150-300 mV at a duty cycle between about fifty and one hundred percent (50-100%) to generate the focused ultrasound having an average peak-to-peak pressure/intensity between about three to nine MegaPascals (3.0-9.0 MPa) for a treatment duration between about thirty and ninety seconds (30-90 s).

19. The device of claim 18, wherein the controller is configured to activate the array in pulses having pulse durations between about twenty five and one hundred milliseconds (25-100 ms) during the treatment duration.

20. (canceled)

21. A method for relieving peripheral nerve pain in a subject, comprising:
placing a contact surface of an ultrasound device against the subject's skin such that an array of transducers of the device are acoustically coupled to the skin;
activating an ultrasound imager of the device to transmit signals from the surface into the subject's body and receive reflected signals from the body to identify a target nerve within the body; and
delivering focused ultrasound from the array from the surface into the body to the target nerve to relieve pain at a frequency of about 1.5 Megahertz using a voltage between about 150-300 mV.

22-32. (canceled)
